(19) United States
(12) Patent Application Publication
(10) Pub. No.: US 2009/0264518 A1
(43) Pub. Date: Oct. 22, 2009

(54) LIQUID KOJI AND QUICK-BREWED MISO-LIKE FOOD

(75) Inventors: Michinari SAKURAI, Kawasaki (JP); Kaname FUJITA, Kawasaki (JP); Naoko HISHIYA, Kawasaki (JP); Yuki OKABE, Kawasaki (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: AJINOMOTO CO., INC, Tokyo (JP)

(21) Appl. No.: 12/428,750

(22) Filed: Apr. 23, 2009

Related U.S. Application Data
(63) Continuation of application No. PCT/JP07/71173, filed on Oct. 24, 2007.

(30) Foreign Application Priority Data
Oct. 26, 2006 (JP) 2006-290685
Apr. 19, 2007 (JP) 2007-110402

(51) Int. Cl. A61K 31/352; C12N 1/14; A61P 19/00; A23L 1/105; A23L 1/36; A23L 1/28

(52) U.S. Cl. 514/456; 435/256.1; 426/7; 426/18; 426/44; 426/61

(57) ABSTRACT

The present invention provides a method for producing a salt-free miso-like food having strong umami and rich flavor with strong taste. As a means to solve the problem, a liquid koji is prepared by culturing an aspergillus in a predetermined amount of lactic acid bacterium culture solution or a supernatant thereof. The thus-obtained liquid koji is added to a food material, and koji making is performed in a hermetically sealed koji-making machine while successively or intermittently supplying disinfected air thereto. Subsequently, a lactic acid bacterium culture solution or a supernatant thereof is mixed into the obtained koji, and optionally a food material is mixed therewith. The obtained mixture is provided as it is or in paste form to give moromi, and the moromi is hydrolyzed in the absence of salt.

### FIELD OF THE INVENTION

The present invention relates to a liquid koji produced while inhibiting the growth of bacteria; a salt-free miso-like food obtained using the liquid koji to make koji stably within a short period of time, and which has strong umami and rich flavor with strong taste; and a bone-metabolism-improving composition.

### BACKGROUND OF THE INVENTION

Traditional brewed, fermented foods, such as miso, are known to take a long time to produce. Recently, working hours have been reduced with an increasing number of holidays, etc., and, in the production field, development of efficient production methods has been desired. In developing an efficient production without making capital investment or the like, the primary bottleneck is the process of koji making. In reducing the production time, reduction in the time of koji-making process brings numerous benefits to the whole processes.

One method to reduce the koji-making time is to increase the koji-making temperature. This method is highly effective, but provides an environment that allows easy proliferation of contaminant microorganisms from the raw materials or facilities, especially bacteria belonging to genus Bacillus, resulting in deterioration in the quality of koji. Such deterioration in the quality of koji leads to deterioration in the quality of the end product, and thus is undesirable.

In the preparation of traditional brewed, fermented foods, salt is added to prevent proliferation of contaminant microorganisms. However, when such a food is applied in seasonings or dressings, the contaminant microorganisms existing in the food may proliferate in such products, causing deterioration of the products.

Further, in recent years, various health functions of brewed, fermented foods have been reported. In particular, demands for miso as a health food are expected, as it contains abundant soy isoflavone and has also been reported as being related to anticancer effects. However, because miso contains a remarkable amount of salt, heavy use thereof is feared to lead to high intake of salt, and the demand is sluggish in reality.

Miso or a food material for miso which requires a reduced koji-making time and is low-salt or salt-free has been demanded for long years. Although some reports have been made on reduction in the koji-making time, reportedly, when koji is made within a short period of time (one to two days), it is difficult to obtain koji with stable quality (Nonpatent Document 1). For example, although a method including promoting germination of seed koji prior to koji making has been disclosed (Patent Document 1), reduction in the actual koji-making time is not mentioned. Further, a method that uses a mixture of koji produced in a reduced koji-making time and normal koji (Nonpatent Document 2) and a method to improve the quantity by using a koji-making auxiliary, adding an enzyme preparation, and so forth (Nonpatent Document 3) have also been reported, but neither has been industrialized.

As prior technique related to the production of low-salt miso or salt-free miso, a method wherein low-salt miso that has been diluted and dialyzed is used to produce a food with a high protein content (Patent Document 2), a method wherein miso is diluted with water to produce desalted miso (Patent Document 3), a method wherein ethanol is added at the time of preparation prior to fermentation (Nonpatent Document 4), etc., have been reported. Further, a method wherein a lactic acid bacterium that produces nisin, a kind of bacteriocin, is inoculated, and then lactic acid fermentation is performed, thereby preparing salt-free miso on a laboratory scale, has been reported. According to this method, reportedly, bacillus or other contaminant bacteria were not detected (Nonpatent Document 5).

However, in the production of miso on an actual industrial scale, even if a nisin-producing lactic acid bacterium is simply added, various microorganisms mixed thereinto from the open air, especially genus Pediococcus, genus Enterococcus or like lactic acid bacteria, remarkably grow during maturation. Such lactic acid bacteria produce lactic acids, whereby the pH is reduced, leading to so-called acidification. Further, nisin does not have antibacterial effects against gram negative bacteria including Escherichia coli or the like (Nonpatent Document 6), and therefore, with only nisin, it is impossible to effectively prevent deterioration due to those bacteria. That is, although preparation of salt-free miso on a laboratory scale is relatively easy in terms of microorganism control, in the production on a commercial scale, control of bacteria is extremely difficult. Accordingly, production of salt-free miso on a commercial scale is believed to be almost impossible.

Nonpatent Document 1 states that the industrial production of salt-free miso is under consideration, but specific production conditions are not disclosed. Needless to say, it nowhere mentions koji making in a hermetically sealed state, which is one feature of the invention.

Moreover, Patent Document 4 discloses a method for making koji using a rotating pressure drum, according to which processing of a koji material (a food material added to koji, such as soybean), i.e., watering, steam boiling, and cooling, is performed in the same apparatus as with koji making, and reports that no bacteria were detected from the koji prepared by such a method. However, in powder seed koji that is used for actual production, contaminant bacterium are often mixed in at a level of 10³⁻⁵ cfu/g, and it seems to be industrially difficult to stably maintain the sterility. Further, Patent Document 4 relates to a method and an apparatus for culturing koji, and nowhere mentions decomposition of moromi in the absence of salt, i.e., production of salt-free miso.

Meanwhile, isoflavone, which is one of the healthy, functional ingredients of miso, exists in soybean and ordinary soybean foods as a glycoside having sugar. After intake, the sugar is removed by intestinal bacteria, whereby the isoflavone is converted into aglycone, which is then absorbed into the body for the first time (Nonpatent Document 7). Therefore, if isoflavone could be taken as an aglycone originally having no sugar, high discovery of isoflavone functions not depending on intestinal bacteria would be expected.

In fermented soybean foods, such as miso, change the aglyconee form of isoflavone is induced by β-glucosidase of an aspergillus during fermentation. However, it is known that because the production is usually proceeded under relatively high-salt conditions, change the aglyconee form of isoflavone within a short period of time is difficult. The ratio of aglyconee form of usual miso products are about 65% in case of rice miso, about 21% in case of white miso, about 58% in case of blended miso, and about 48% in case of barley miso (Nonpatent Document 8). A fermented soybean food with a high ratio of aglyconee form has not been achieved.

The ratio of aglyconee form of bean miso produced using soybean as a koji material is about 90% (Nonpatent Document 8); however, long-term maturation (about two years) will be required (Nonpatent Document 9). Achievement of high ratio of aglyconee form by short-term maturation has not been reported.

Patent Document 1

Patent Document 2

Patent Document 3

Patent Document 4

Nonpatent Document 1

Takafumi Unoki, Kagoshima-Ken Kogyo Gijutu Center Kenkyu Houkoku (Research Report, Kagoshima Industrial Technology Center) No. 16 (2002)

Nonpatent Document 2

Hayade Akio, Shinshu-Miso Kenkyu Houkoku (Shinshu-Miso Research Report), 43, pp. 58-60 (1993)

Nonpatent Document 3

Takashi Akimoto, Miso no Kagaku to Gijutu (Science and Technology of Miso), 39, p. 355-363 (1991)

Nonpatent Document 4

Satoru Watanabe, Hokuriku Nogyo Kenkyu Seika Joho (Information on Hokuriku Agricultural Research Results), 15, pp. 169-170 (1999)

Nonpatent Document 5

Takeo Kato, Nihon Jozo Kyokai Shi (Journal of Japan Brewing Society), 97, pp. 615-623 (2002)

Nonpatent Document 6

Toshio Matsuda, Shokuhin no Hikanetsu Sakkin Oyo Handbook (Comprehensive Handbook of Nonthermal Food Sterilization), p. 187

Nonpatent Document 7

Setchell K D, et al., Am. J. Clin. Nutr., 76 2, pp. 447-453 (2002)

Nonpatent Document 8

Toshiya Toda, et al., FFI journal, 172, pp. 83-89 (1997)

Nonpatent Document 9

Kihara, et al., Nihon Shoyu Kenkyujo Zasshi (Journal of Japan Soy Sauce Research Institute), 17 1, pp. 1-4 (1991)

### DISCLOSURE OF THE INVENTION

An object of the invention is to provide 1) a liquid koji wherein the growth of bacteria is inhibited, 2) a low-salt or salt-free quick-brewed miso-like food having strong umami and rich flavor with strong taste obtained using the liquid koji, and 3) a composition effective in improving bone metabolism.

The present inventors conducted extensive research to solve the above problems, and as a result found a method of culturing an aspergillus in 5 to 500 times its amount of lactic acid bacterium culture solution. They also found that when the obtained aspergillus culture solution is added to a food material, followed by koji making in a hermetically sealed koji-making machine for 16 to 40 hours while successively or intermittently supplying disinfected air thereto, then mixing a lactic acid bacterium culture solution or a supernatant thereof into the thus-obtained koji, optionally further mixing therewith at least one kind of food material in an amount 0.01 to 50 times the weight of the koji, and providing the mixture as it is or in paste form to give moromi, a novel salt-free miso-like food having strong umami, rich flavor, and strong taste can be obtained. They further found that a novel salt-free miso-like food produced using soybean as a koji material is effective in improving bone metabolism. Specifically, the invention includes the following items.

(1) A liquid koji obtainable by culturing an aspergillus in 5 to 500 times its amount of lactic acid bacterium culture solution or a supernatant thereof.
(2) A liquid koji according to (1), wherein the aspergillus is cultured under conditions of culture temperature of 20 to 45° C. and culture time of 6 to 30 hours.
(3) A liquid koji according to (1), wherein the lactic acid bacterium is a bacteriocin-producing bacterium.
(4) A liquid koji according to (3), wherein the bacteriocin-producing lactic acid bacterium is a nisin-producing lactic acid bacterium.
(5) A quick-brewed miso-like food comprising a step 1 of adding the liquid koji of anyone of items (1) to (4) to a food material; a step 2 of making koji in a hermetically sealed koji-making machine at 20 to 45° C. for 16 to 40 hours while successively or intermittently supplying disinfected air thereto; a step 3 of mixing a lactic acid bacterium culture solution or a supernatant thereof into the thus-obtained koji, and optionally further mixing therewith at least one kind of food material in an amount 0.01 to 50 times the weight of the koji; a step 4 of providing the mixture as it is or in paste form to give moromi; and a step 5 of hydrolyzing the moromi substantially in the absence of salt.
(6) A quick-brewed miso-like food according to (5), wherein the lactic acid bacterium is a bacteriocin-producing bacterium.
(7) A quick-brewed miso-like food according to (6), wherein the bacteriocin-producing lactic acid bacterium is a nisin-producing lactic acid bacterium.
(8) A quick-brewed miso-like food according to (5), wherein the food material used in the step 1 and/or the step 3 of (5) is at least one kind selected from the group consisting of rice, barley, soybean, and soy germ.
(9) A quick-brewed miso-like food according to (5), wherein the food material used in the step 1 of (5) is soybean and the food material used in the step 3 is rice.
(10) A quick-brewed miso-like food according to (5), wherein the quick-brewed miso-like food has a soy isoflavone aglyconeization percentage of 80 to 100%.
(11) A bone-metabolism-improving composition comprising a quick-brewed miso-like food having a soy isoflavone aglyconeization percentage of 80 to 100%.
(12) A bone-metabolism-improving composition comprising a quick-brewed miso-like food having an aglyconeization percentage of 80 to 100% obtained through a step 1 of adding a liquid koji to soybean; a step 2 of making koji in a hermetically sealed koji-making machine at 20 to 45° C. for 16 to 40 hours while successively or intermittently supplying disinfected air thereto; a step 3 of mixing a lactic acid bacterium culture solution or a supernatant thereof into the thus-obtained koji, and optionally further mixing therewith at least one kind of food material in an amount 0.01 to 50 times the weight of the koji; a step 4 of providing the mixture as it is or in paste form to give moromi; and a step 5 of hydrolyzing the moromi substantially in the absence of salt.
(13) A bone-metabolism-improving composition according to (12), wherein the liquid koji is obtained by culturing an aspergillus in 5 to 500 times its amount of lactic acid bacterium culture solution or a supernatant thereof.
(14) A bone-metabolism-improving composition according to (13), wherein the aspergillus is cultured under conditions of culture temperature of 20 to 45° C. and culture time of 6 to 30 hours.
(15) A bone-metabolism-improving composition according to (13), wherein the lactic acid bacterium is a bacteriocin-producing bacterium.
(16) A bone-metabolism-improving composition according to (15), wherein the bacteriocin-producing lactic acid bacterium is a nisin-producing lactic acid bacterium.
(17) A bone-metabolism-improving composition according to (12), wherein the food material used in the step 1 and/or the step 3 of (12) is at least one kind selected from the group consisting of rice, barley, soybean, and soy germ.
(18) A bone-metabolism-improving composition according to (12), wherein the food material used in the step 1 of (12) is soybean and the food material used in the step 3 is rice.
(19) A bone-metabolism-improving composition according to (11), wherein the bone-metabolism-improving composition is an eatable or a drinkable.

According to the invention, because the production proceeds in the absence of salt (or substantially in the absence of salt), the enzyme activity of protease, peptidase, or the like in the aspergillus is not inhibited by salt, and it thus is possible to produce a novel salt-free miso-like food having strong umami, rich flavor, and strong within an extremely shorter fermentation and maturation period compared with the case of ordinary miso. Further, among novel salt-free miso-like foods, a novel salt-free miso-like food which is obtained using soybean as a koji material and 80 to 100% of soy isoflavone change aflycone form is effective in improving bone metabolism, and thus is expected to be applied to the prevention, treatment, and alleviation of osteoporosis.

The miso-like food of the invention is salt-free (or substantially salt-free), and accordingly has advantages in that it gives a healthful image and also is safe because no contaminant bacteria exist therein, and further, again, the production time can be greatly reduced.

### BRIEF DESCRIPTION OF THE DRAWINGS

FIG. 1 shows the amount of deoxypyridinoline in urine of rats administered the composition of the invention (test sample) and a composition containing a non-fermented soybean product (contrast sample).

FIG. 2 shows the tibial cancellous bone density of rats administered the composition of the invention (test sample) and a composition containing a non-fermented soybean product (contrast sample).

FIG. 3 shows the Ca content of tibia per body weight of rats administered the composition of the invention (test sample) and a composition containing a non-fermented soybean product (contrast sample).

FIG. 4 shows the total isoflavone amount in urine of rats administered the composition of the invention (test sample) and a composition containing a non-fermented soybean product (contrast sample).

### BEST MODE FOR CARRYING OUT THE INVENTION

In the invention, any usual lactic acid bacteria may be used for the lactic acid bacterium culture solution for culturing an aspergillus. For example, culture solutions of bacteria belonging to genus Lactobacillus, genus Lactococcus, genus Weissella, etc., can be used. Among these, a bacteriocin-producing lactic acid bacterium is preferable.

Further, among bacteriocins, nisin can be especially preferred for its broad antimicrobial spectrum, and therefore, a nisin-producing lactic acid bacterium culture solution is the most preferable. The kind of nisin produced by the lactic acid bacterium may be any of nisin A, nisin Z, and analogs thereof.

Specifically, for example, Lactococcus lactis AJ110212 (FERM BP-8552) that allows high production of nisin Z can be used. The L. lactis AJ110212 strain has been deposited at the National Institute of Advanced Industrial Science and Technology, International Patent Organism Depository (Tsukuba Central 6, 1-1, Higashi 1-chome, Tsukuba-shi, Ibaraki, 305-8566, JAPAN), on Nov. 19, 2003, under an Accession Number of FERM BP-8552 as an international depositary under the Budapest Treaty.

Naturally, in order to prevent contamination with microorganisms and growth thereof, the lactic acid bacterium culture solution or a supernatant thereof used preferably contains bacteriocin, and the activity thereof is preferably high.

The nisin activity should be 200 IU/ml or more. Usually, a culture solution having a nisin activity of 200 to 2000 IU/ml or a supernatant thereof is used in an amount 5 to 500 times the weight of the aspergillus, preferably 10 to 200 times the weight, and more preferably 20 to 100 times the weight.

When the amount is less than 5 times the weight, it is difficult to uniformly disperse an aspergillus in the lactic acid bacterium culture solution, resulting in lower antimicrobial activity, and contamination with bacteria cannot be avoided. When the amount is more than 500 times the weight of the koji, the aspergillus concentration in the culture solution is shorted for using the following step, whereby the koji making step is delayed.

The aspergillus used is preferably, but is not limited to, one that is capable of highly decomposing the material protein into an amino acid or peptide, and imparting the resulting novel miso-like food with strong umami, rich flavor, and strong. For example, Aspergillus oryzae, Aspergillus sojae, and like bacteria belonging to genes Aspergillus used in the production of miso can be used.

A liquid koji is prepared from the culture of an aspergillus in a lactic acid bacterium culture solution or a supernatant thereof in amount of 5 to 500 parts by weight of the aspergillus, but conditions for the culture are not limited. Specifically, usual conditions for culturing aspergilli may be directly used as the aspergillus culture conditions. For example, the culture temperature is preferably 20 to 45° C. This is because the growth of aspergilli is remarkably hampered at temperatures of not more than 20° C. or not less than 45° C. When the culture is performed at 20 to 45° C., the culture time may usually be 6 to 30 hours. When the culture time is less than 6 hours, germination of bacteria is barely observed, and the koji-making time cannot be reduced. Further, when the culture time is 30 hours or more, the fungal threads grow well, and it becomes difficult to mix a culture solution into a food material in the below-mentioned step 1.

Now, the liquid koji prepared by culturing an aspergillus in a lactic-acid culture solution or a supernatant thereof is added to a food material called a koji material, and then mixed (step 1). The amount of liquid koji to be used is not limited, and is usually 0.1 to 10 wt %, preferably 0.5 to 5 wt %, relative to the koji material.

Subsequently, the mixture is charged into a koji-making machine which can be hermetically sealed and to which disinfected air can be supplied. A koji-making machine which can be hermetically sealed and to which disinfected air can be supplied may be an apparatus having a function of supplying disinfected air into the koji-making machine, and having a structure that allows the inside of the koji-making machine to be shut off from the open air. For example, a rotating drum koji-making machine can be mentioned. However, a koji-making machine having a structure for supply of disinfected air thereinto and provided with an openable/closable lid that provides a hermetically sealed state is simpler in structure, less expensive, and thus is more preferable.

As a koji material, a food material used in the preparation of usual miso may be used, such as soybean, rice, barley, soy germ, or the like. If necessary, these materials may have been subjected to water immersion, peeling, shredding, or a like pretreatment, or steam boiling, roasting, or a like heating treatment.

One feature of the invention is that koji is prepared in a hermetically sealed space while successively or intermittently supplying disinfected air thereto (step 2). As a method for disinfecting air, a method using a filter capable of collecting 0.3-μm or larger dust 99.97% or more, for example, a HEPA filter, may be employed. The ventilation method is not limited, and internal ventilation, surface ventilation, and the like may be employed.

Where possible, use of a koji-making machine that cannot be sealed hermetically, such as a rotating-disk koji-making machine, a stationary-ventilation koji-making machine, or the like, should be avoided, because such a koji-making machine may allow contamination with microorganisms from the outside air. In particular, bacteria belonging to genus Pediococcus, bacteria belonging to genus Enterococcus, or like lactic acid bacteria may be mixed in which results in the formation of lactic acids during maturation, leading to so-called acidification.

After the mixture of a koji material and a liquid koji (aspergillus culture solution) is charged into a koji-making machine, in the state where the koji-making machine is hermetically sealed, culture is performed at 25 to 40° C. for 16 to 40 hours, and preferably at 34 to 40° C. for 22 to 31 hours, while supplying disinfected air successively or intermittently thereto, thereby giving koji (step 2). A koji-making temperature of more than 40° C. reduces enzyme activity necessary for the decomposition of the material protein, while a temperature of less than 25° C. hampers the growth of the aspergillus. In either case, an enzyme necessary for the decomposition of the material protein cannot be sufficiently obtained, and a novel miso-like food satisfactorily having umami, rich flavor, and strong taste cannot be provided.

Further, when the koji-making time is less than 16 hours, it is difficult for the aspergillus to grow sufficiently, and a novel miso-like food satisfactorily having umami, rich flavor, and strong taste cannot be provided. When the koji-making time is more than 40 hours, the enzyme activity necessary for the decomposition of the material protein is reduced. As result, umami, rich flavor, and strong cannot be satisfactorily provided, and further, bitterness is imparted.

Subsequently, a lactic acid bacterium culture solution or a supernatant thereof is added to the obtained koji in an amount such that the moisture content of the resulting moromi be 35 to 60%, and more preferably 40 to 50%. Specifically, the amount may be 0.01 to 5 times the weight of the koji, and more preferably 0.1 to 1.0 times the weight.

Optionally, at least one kind of food material is added in an amount 0.01 to 50 times the weight of the koji, and moromi is thus formed (step 3). The food materials to be added to the koji to form moromi (called moromi materials) are also those used in the preparation of usual miso, such as soybean, rice, barley, soy germ, and the like. In this case, “optionally” means that when bean koji whose koji material is soybean is used, it may be unnecessary to add soybean again in the preparation of moromi. That is, when soybean is used as a koji material, koji is prepared from a mixture of soybean, a lactic acid bacterium culture solution or a supernatant thereof, and an aspergillus, and a lactic acid bacterium culture solution or a supernatant thereof is further added thereto, thereby giving moromi.

Further, soybean, rice, barley, soy germ, or a like food material may also be added to the koji. In such a case, the food material may have been previously steam boiled or roasted. For example, when rice koji or barley koji whose koji material is rice or barley is used, steam boiled or roasted soybean may be added. When bean koji is used, steam boiled or roasted barley or rice may be added. An extract of such a vegetable material or a specific component thereof may also be used. Examples thereof include soybean extract, rice starch, barley bran, etc.

Further, according to the kind and the amount of the added food material, color, taste, flavor, and the like can be controlled. For example, addition of rice imparts sweetness. In particular, when soybean is used as a koji material, and rice is added as a food material in the step 3, the resulting product has excellent taste and flavor.

The order of adding a lactic acid bacterium culture solution or a supernatant thereof and a food material such as rice, barley, or the like to koji may be arbitrarily determined. Food materials may be added to koji before, after, or at the same time as the addition of a lactic acid bacterium culture solution or a supernatant thereof.

As mentioned above, a lactic acid bacterium culture solution or a supernatant thereof is added usually in an amount of 0.01 to 5 parts by weight of the koji weight. When the amount is less than 0.01 times the weight, suppression of the growth of microorganisms cannot be achieved. When the amount is more than 5 times the weight, these results in insufficient decomposition of protein, and a novel miso-like food satisfactorily having umami, rich flavor, and strong cannot be provided.

Subsequently, the mixture of koji, a nisin lactic acid bacterium culture solution or a supernatant thereof, and optionally at least one kind of food material is ground in a chopper or the like to a paste, thereby giving moromi (step 4). The moromi is stored in the absence of salt (or substantially in the absence of salt) at a temperature maintained at 20 to 50° C., preferably 20 to 45° C., and more preferably 30 to 40° C., fermented and matured for 1 to 50 days, preferably 4 to 30 days, more preferably 7 to 21 days, and still more preferably 10 to 17 days, and hydrolyzed (step 5).

A temperature of less than 20° C. results in insufficient decomposition of protein, and a novel miso-like food satisfactorily having umami, rich flavor, and strong cannot be provided. A temperature of more than 50° C. induces reaction between sugar and amino acid contained in the moromi, which imparts browning odor, burnt odor, and bitterness to the resulting novel miso-like food, and thus is not preferable.

The same tendency is also observed with respect to the number of days for maturation. A maturation time of less than 24 hours results in insufficient decomposition of protein, and a novel miso-like food satisfactorily having umami, rich flavor, and strong taste can not be provided. A maturation time of more than 50 days induces reaction between sugar and amino acid contained in the moromi, which imparts browning odor, burnt odor, and bitterness to the resulting novel miso-like food, and thus is not preferable. Further, for the prevention of contamination with bacteria, it is preferable that, for example, moromi be packaged in a laminated pouch, a plastic container, or the like, so that hydrolyzation of moromi is performed in a hermetically sealed system.

Subsequently, if necessary, the matured and hydrolyzed moromi is heated at 50 to 130° C. for 1 to 150 minutes. The purpose of heating is sterilization and also deactivation of protease and like enzymes contained in moromi to prevent change in quality under preservation. The method of heating is not limited. For example, a double tube heater and a multi-tubular heater used for pasteurization of miso can be used. It is also possible to package moromi in a pouch or the like, and heat the same in a water bath, etc. A temperature of less than 50° C. results in insufficient sterilization and enzyme deactivation. While a temperature of more than 130° C. result in imparts browning odor, burnt odor, and bitterness, and thus is not preferable. The same tendency is also observed with respect to time. A time of less than 1 minute results in insufficient sterilization and enzyme deactivation. While a time of more than 150 minutes results in imparts browning odor, burnt odor, and bitterness, and thus is not preferable.

The miso-like food obtained by the method of the invention can be used in the original paste form, and may also be dried with a spray dryer, a drum dryer, a vacuum drum dryer, a freezing dryer, or the like, and used in the form of powder. In the embodiment of the novel miso-like food obtained by the method of the invention, it may be mixed into various eatables and drinkables at the time of production or processing thereof, used as a mixture with various liquid, granular, or powdery seasonings, taken as it is, etc.

The miso-like food obtained by the method of the invention is salt-free (or substantially salt-free), and has strong umami, rich flavor, and strong taste, with reduced stuffy odor and astringent taste. Such a miso-like food can thus be widely applied not only to miso soup but also to various kinds of eatables and drinkables. Although miso cannot be taken in large quantities because of its high salt content. because the miso-like food of the invention is salt-free (or substantially salt-free), the healthy, functional ingredients contained in soybean can be taken abundantly. Further, the quick-brewed miso-like food prepared using soybean as a koji material has ratio of isoflavone aglyconee of 80 to 100%, thus provides an advantage that allows easy absorption of isoflavone, and is expected to lead to high expression of isoflavone functions, specifically, a bone-metabolism-improving function.

The bone-metabolism-improving composition of the invention is characterized in comprising a quick-brewed miso-like food having ratio of soy isoflavone aglyconee of 80 to 100%. Insofar as the quick-brewed miso-like food has ratio of soy isoflavone aflycone of 80 to 100%, a composition produced by any production method may be used as the bone-metabolism-improving composition of the invention.

However, the quick-brewed miso-like food wherein the ratio of soy isoflavone aflycone is 80 to 100% is preferably prepared by the above method, i.e., a method comprising a step 1 of adding a liquid koji to soybean, a step 2 of making koji in a hermetically sealed koji-making machine at 20 to 45° C. for 16 to 40 hours while successively or intermittently supplying disinfected air thereto, a step 3 of mixing a lactic acid bacterium culture solution or a supernatant thereof into the thus-obtained koji, and optionally further mixing therewith at least one kind of food material in an amount 0.01 to 50 times the weight of the koji, a step 4 of providing the mixture as it is or in paste form to give moromi, and a step 5 of hydrolyzing the moromi substantially in the absence of salt.

As mentioned above, the liquid koji is preferably prepared by culturing an aspergillus in 5 to 500 parts by weight of lactic acid bacterium culture solution or a supernatant thereof preferably under culture conditions of culture temperature of 20 to 45° C. and culture time of 6 to 30 hours, the lactic acid bacterium is preferably a bacteriocin-producing bacterium, the bacteriocin-producing lactic acid bacterium is preferably a nisin-producing lactic acid bacterium, the food material used in the step 1 and/or the step 3 is preferably at least one kind selected from the group consisting of rice, barley, soybean, and soy germ, and the food material used in the step 1 is preferably soybean and the food material used in the step 3 is preferably rice.

The bone metabolism composition of the invention may be taken as it is, and may also be processed into juice, soup, miso soup, cookies, dumplings, or like various eatables and drinkables.

### EXAMPLES

Hereafter, the prevent invention is explained in further detail through the Examples. Needless to say, the invention is not limited to these examples.

### Example 1

A 2.5-g quantity powder seed koji (manufactured by BIOC) was mixed with 100 g of culture solution of Lactococcus lactis AJ110212 (FERM BP-8552), which is a high nisin Z producing lactic acid bacterium, and cultured at 35° C. for 18 hours to prepare a liquid koji. The nisin activity was then 1350 IU/ml.

As control, 2.5 g of powder seed koji was mixed with 100 g of water, and cultured at 35° C. for 18 hours in the same manner.

The number of contaminant bacteria and the germination rate after completion of the culture are shown in Table 1. A shown in Table 1, in the seed koji cultured in water, bacteria belonging to genus Bacillus and like contaminant bacteria were detected, while no bacteria other than lactic acid bacteria were detected in the seed koji cultured in a lactic acid bacterium culture solution. With respect to germination rate, no difference was observed between the water culture and the culture in a lactic acid bacterium culture solution.

TABLE 1

| Solution for Seed Koji | Kind of Contaminant Bacteria: | Germination |
|---|---|---|
| Culture | Number of Bacteria (cfu/ml) | rate |
| Water | Bacillus sp.: 1 × 10⁴ | 45.5% |
| | Staphylococcus sp.: 2 × 10⁶ | |
| | Enterobacter sp.: 3 × 10⁴ | |
| Lactic Acid Bacterium | <20 | 42.5% |
| Culture Solution | | |
| (L. lactis AJ110212) | | |

### Example 2

A 1-g quantity powder seed koji (manufactured by BIOC) was mixed with 40 g of the same culture solution of Lactococcus lactis AJ110212 (FERM BP-8552), which is a high nisin Z producing lactic acid bacterium, as used in Example 1, and cultured at 35° C. for 18 hours to prepare a liquid koji. The nisin activity was 1250 IU/ml.

Simultaneously, the bacteria shown in Table 2, which are contaminant bacteria derived from seed koji, were added at 1×10² bacteria per gram of seed koji to forcibly make the culture contaminated. The number of contaminant bacteria after completion of the culture is shown in Table 2. As shown in Table 2, none of the added bacteria were detected.

TABLE 2

| | Number of Bacteria (cfu/ml) |
|---|---|
| Kind of Contaminant Bacteria | after 18-Hour Culture |
| Bacillus sp. | <20 |
| Staphylococcus sp. | <20 |
| Enterobacter sp. | <20 |

### Example 3

A 0.005-kg quantity powder seed koji (Aspergillus oryzae, manufactured by BIOC) was mixed with 0.2 kg of the same culture solution of Lactococcus lactis AJ110212 (FERM BP-8552) (nisin activity: 1500 IU/ml), which is a high nisin Z producing lactic acid bacterium, as used in Example 1, and cultured at 35° C. for 18 hours to prepare a liquid koji.

Meanwhile, 5.0 kg of soybean was immersed in water to absorb water, and then steam boiled in a digester at 114° C. for 30 minutes. The above culture solution and the steam boiled soybean were mixed, charged into a hermetically sealable koji-making machine, and then subjected to koji making in a hermetically sealed koji-making machine at 35° C. with a humidity of 90% for 16 to 31 hours. For the purpose of removing the fermentation heat during koji making, the machine was continuously ventilated with air disinfected through a HEPA filter.

Subsequently, 5.4 kg of rice and soy germ that have been steam boiled at 100° C. for 50 minutes and then cooled to normal temperature was added thereto, and then 1861 g of the same culture solution of lactic acid bacteria (Lactococcus lactis AJ110212, FERMBP-8552) was added. The mixture was then ground in a chopper to a paste. The paste was placed in a laminated pouch to an amount of 5 kg per pouch. The pouches were stored at 35° C. for 14 days, and then miso-like foods were prepared.

The miso-like foods obtained through the above processes were microbiologically analyzed. Further, sensory evaluation was carried out by three expert sensory evaluation panelists. Using a 10% aqueous solution, the sensory evaluation was made on a scale of 1 to 5, with 3 being the pass mark. Evaluation criteria are umami (heaviness), richness of flavor, sourness, and bitterness. The samples were evaluated comprehensively based on these criteria. The results are shown in Table 3. As a result, samples with a koji-making time of 16 hour or more made passing scores, and those with 22 hours or more made high scores.

TABLE 3

| Koji-Making Time (hour) | 16 | 19 | 22 | 25 | 28 | 31 |
|---|---|---|---|---|---|---|
| Sensory Score | 3.0 | 3.5 | 4.0 | 5.0 | 5.0 | 5.0 |

### Example 4

A 40-g quantity powder seed koji (manufactured by BIOC) was mixed with 2 kg of the same culture solution of Lactococcus lactis AJ110212 (FERM BP-8552) (nisin activity: 850 IU/ml), which is a high nisin Z producing lactic acid bacterium, as used in Example 1, and cultured at 35° C. for 18 hours to prepare a liquid koji. Meanwhile, 40 kg of soybean was immersed in water to absorb water, and then steam boiled in a digester at 114° C. for 30 minutes. The above culture solution and the steam boiled soybean were mixed, charged into a hermetically sealable laboratory koji-making machine, and then subjected to koji making in a hermetically sealed koji-making machine at 35° C. with a humidity of 90% for 25 hours. For the purpose of removing the fermentation heat during koji making, the machine was continuously ventilated with air disinfected through a HEPA filter. The number of contaminant bacteria in the thus-obtained koji was checked.

Subsequently, 55 kg of rice and soy germ that have been steam boiled at 100° C. for 50 minutes and then cooled to normal temperature was added thereto, and then 3 kg of the same culture solution of lactic acid bacteria (Lactococcus lactis AJ110212, FERM BP-8552) was added. The mixture was then ground in a chopper to a paste. The paste was placed in a laminated pouch to an amount of 5 kg per pouch. The pouch was stored at 35° C. for 14 days, and then a miso-like food was prepared.

The miso-like food obtained though the above processes and commercial miso was analyzed for the amount of isoflavone (glycoside, aglycone). Further, sensory evaluation was carried out by two expert sensory evaluation panelists. A 5% aqueous solution was used in the sensory evaluation. The salt concentration thereof was adjusted to 1%. The results are shown in Table 4. The criteria for the sensory evaluation are the same as in Example 3.

As a result of evaluation, umami of commercial miso was weak, and its flavor was not so rich or heavy, while the flavor of the novel miso-like food was rich and heavy. Further, with respect to isoflavone, the proportion of isoflavone aglyconee, which is believed to be highly absorbable, was 35% in the commercial miso, while the proportion was as high as 99% in the novel miso-like food.

TABLE 4

| | | Amount of Isoflavone | |
|---|---|---|---|
| | | Novel Miso- | Commercial |
| | | Like Food | Rice Miso |
| Total Isoflavone | mg/100 g | 126 | 73 |
| Aglyconee | mg/100 g | 125 | 26 |
| Glycoside | mg/100 g | 1 | 48 |
| Aglyconee/Total Isoflavone | (%) | 99 | 35 |

Sensory Evaluation

| | |
|---|---|
| Novel Miso- | Commercial |
| Like Food | Rice Miso |

| | | |
|---|---|---|
| Sensory Score | 5.0 | 3.5 |

### Example 5

A 21-kg quantity soybean was immersed in water to absorb water, and then steam boiled in a digester at 114° C. for 30 minutes. The steam boiled soybean was fed into a hermetically sealed koji-making machine, and 420 g of culture solution of Lactococcus lactis AJ110212 (FERM BP-8552) (nisin activity: 3000 IU/ml) and 21 g of seed koji (Aspergillus oryzae, manufactured by BIOC) were mixed therewith. Koji making was then performed at 30° C. for 43 hours.

For removing of the fermentation heat during koji making, air disinfected through a HEPA filter was used.

To the obtained koji was added 6.79 kg of culture solution of Lactococcus lactis AJ110212 (FERMBP-8552) (nisin activity: 3000 IU/ml). The mixture was then ground in a chopper to a paste. The paste was placed in a laminated pouch to an amount of 1 kg per pouch. The pouch was stored at 30° C. for 7 days, and then heated at 80° C. for 55 minutes. To 32 kg of the paste were added as an excipient 8.8 kg of dextrin and a proper quantity of water, and then mixed in an inclined-shaft kneader. The obtained liquid preparation was dried in a vacuum drum dryer to give about 24 kg of composition containing the miso-like food material of the invention. The miso-like food material had been prepared under salt-free conditions.

The ratio of aglyconee isoflavone of the thus-obtained miso-like food material was 100%.

### Example 6

Using the composition of the invention prepared in the above Example 5 as a test sample, the composition was mixed into feed in a proportion of 59%, and administered to ovariectomized rats (postmenopausal osteoporosis model) to examine whether the long-term intake of the composition of the invention would improve the bone metabolism of the ovariectomized rats.

A non-fermented soybean product obtained from material soybean braised in water and processed to a paste was pulverized and used as control. Specifically, 28 kg of soybean was immersed in water to absorb water, then steam boiled in a digester at 114° C. for 40 minutes, and ground in a chopper to a paste. To 32 kg of the obtained paste were added as an excipient 8.8 kg of dextrin and a proper quantity of water, and then mixed with an inclined-shaft kneader. The obtained liquid preparation was dried in a vacuum drum dryer to give about 24 kg of composition containing a powdery non-fermented soybean product (control sample) (Ratio of aglycone isoflavone form: 5%).

The test method is shown below in detail. Eight-week-old female SD rats were subjected to ovariectomy (OVX). Rats with a favorable postoperative course were divided to avoid a difference in body weight. The compositions were mixed with feed and administered to the rats for eight weeks; the composition of the invention (test sample) was administered to one group as Test, and a composition containing a non-fermented soybean product (contrast sample) was administered to the other group as Control.

Further, a group of rats that underwent sham was given as a Normal group, and a composition containing a non-fermented soybean product (contrast sample) mixed with feed was administered thereto.

The feed contains the composition of the invention (test sample) in a proportion of 59% and the composition containing a non-fermented soybean product (contrast sample) in a proportion of 61%, so that the isoflavone content of the feed is 228 μmol/100 g (in terms of in rats' intake, about 120 μmol/kg BW: BW means Body Weight). It was identified by analysis that isoflavone in the composition of the invention (test sample) was 100% aglycone, while isoflavone in the composition containing a non-fermented soybean product (contrast sample) was 5% aglycone, the remaining 95% being glycoside.

In the feed, the lipid content was set at 11%, the calcium content at 0.5%, and the phosphorus content at 0.3%. The total nitrogen content was also adjusted the same among all the groups. No feed has added thereto substances that promote or inhibit calcium absorption.

OVX is frequently reported to cause excessive acceleration of bone resorption. Then, deoxypyridinoline in urine, which is a bone resorption marker, was determined.

Specifically, feed containing the composition of the invention (test sample) was administered to OVX rats, and feed containing the composition containing a non-fermented soybean product (contrast sample) was administered to OVX rats and Sham rats, for three weeks. Three weeks later, urine was collected for 24 hours. Each group consists of ten rats.

Using the obtained urine, the amount of deoxypyridinoline was determined using a commercial kit, Osteolinks (DPD) (manufactured by SUMITOMO PHARMA BIOMEDICAL). The obtained values were corrected with the amount of creatinine in urine.

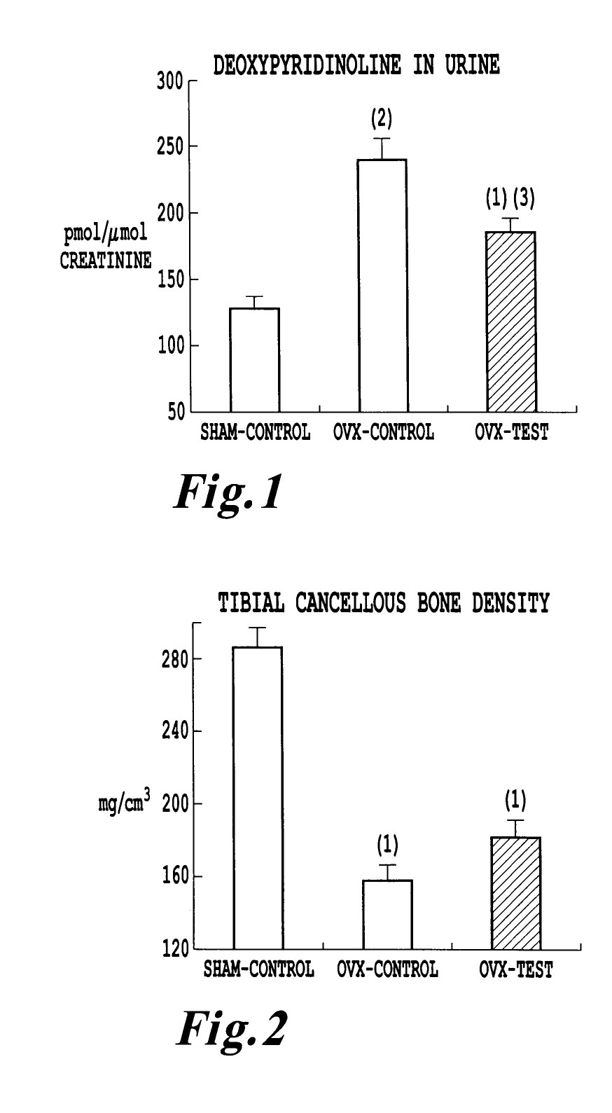

As a result, the amount of deoxypyridinoline in urine in the OVX rats significantly increased as compared with the Sham rats. Moreover, of the OVX rats, the increase in the amount of deoxypyridinoline was significantly inhibited in the Test rats as compared with the Control rats (FIG. 1). More specifically, as compared with the Sham rats, the OVX-treated Test rats had a critical rate of 0.01 or less, which indicates a significant difference (corresponding to (1) in FIG. 1). Further, the OVX-treated Control rats had a critical rate of 0.0001 or less as compared with the Sham rats, which indicates a significant difference (corresponding to (2) in FIG. 1). Further, as compared with the OVX-treated Control rats, the OVX-treated Test rats hada critical rate of 0.01 or less, which indicates a significant difference (corresponding to (3) in FIG. 1). The statistic is means±SEM.

This confirms that as compared with the composition containing a non-fermented soybean product, which is a control sample, the composition of the invention (test sample) significantly inhibits excessive acceleration of bone resorption caused by OVX.

Feed containing the composition of the invention (test sample) was administered to OVX rats, and feed containing the composition containing a non-fermented soybean product (contrast sample) was administered to OVX rats and Sham rats, for eight weeks. Subsequently, the right tibia of the rats was extracted, and the cancellous bone density at the end of the tibia was determined by a pQCT method using XCT Resarch SA+ (manufactured by STRATEC). Each group consists of ten rats.

As a result, the tibial cancellous bone density in the OVX rats significantly decreased as compared with the Sham rats. However, of the OVX rats, the decrease in bone density was smaller in the Test rats than in the Contrast rats (FIG. 2).

More specifically, as compared with the Sham rats, both the OVX-treated Control rats and Test rats had a critical rate of 0.0001 or less, which indicates a significant difference (corresponding to (1) in FIG. 2). The statistic is means±SEM.

Feed containing the composition of the invention (test sample) was administered to OVX rats, and feed containing the composition containing a non-fermented soybean product (contrast sample) was administered to OVX rats and Sham rats, for eight weeks. Subsequently, the left tibia of the rats was extracted, dried (110° C., 12 hours), ashed (680° C., 24 hours), and dissolved in dilute hydrochloric acid. The calcium (Ca) weight of the thus-obtained samples was determined using a commercial kit, C-Test Wako (manufactured by WAKO PURE CHEMICAL INDUSTRIES). Each group consists of ten rats.

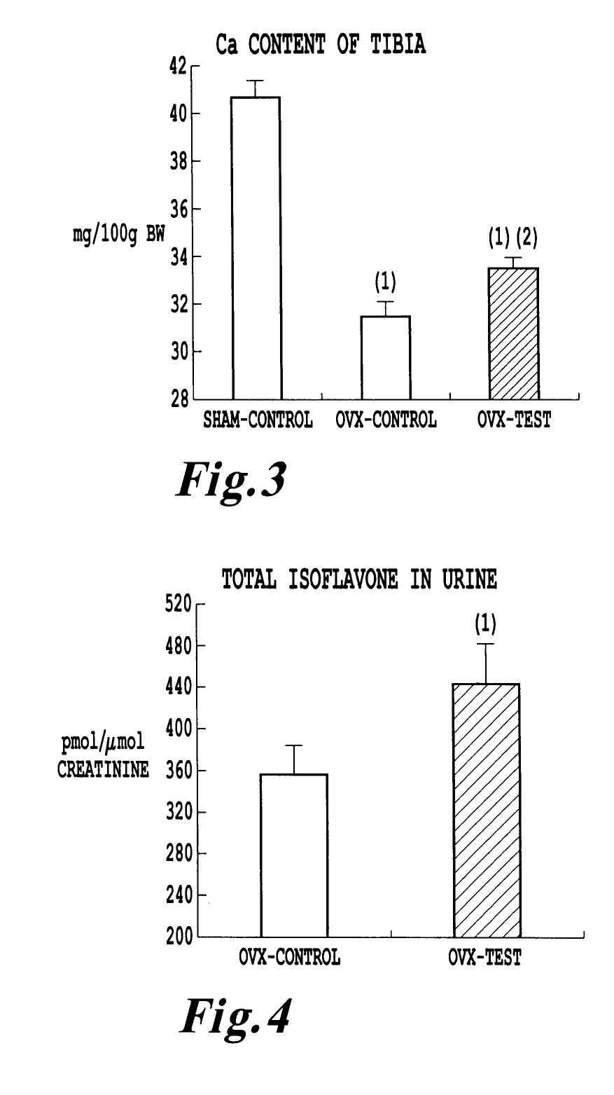

As a result, the Ca content of the tibia in the OVX rats significantly decreased as compared with the Sham rats. However, of the OVX rats, the decrease in the Ca content of the tibia was significantly inhibited in the Test rats as compared with the Control rats (FIG. 3). The values in the figure are values per weight.

More specifically, as compared with the Sham rats, both the OVX-treated Control rats and Test rats had a critical rate of 0.0001 or less, which indicates a significant difference (corresponding to (1) in FIG. 3). Further, the OVX-treated Test rats had a critical rate of 0.05 or less as compared with the OVX-treated Control rats, which indicates a significant difference (corresponding to (2) in FIG. 3). The statistic is means±SEM.

The above data strongly suggest that as compared with the composition containing a non-fermented soybean product, which is a contrast sample, the composition of the invention inhibits excessive bone resorption resulting from OVX and has bone-metabolism-improving effects, such as improvement of bone density and calcium content of bone, etc.

Feed containing the composition of the invention (test sample) was administered to OVX rats, and feed containing the composition containing a non-fermented soybean product (contrast sample) was administered to OVX rats and Sham rats, for eight weeks. Each group consists of ten rats. After the eight-week administration, urine was collected, and the amount of isoflavone was analyzed. That is, because isoflavone exists mainly as a conjugate in the living body, the isoflavone was first hydrolyzed with beta-glucuronidase, then extracted with ethyl acetate, and analyzed by column-switching HPLC. The obtained values were corrected with the amount of creatinine in urine.

As a result, the total amount of isoflavone in urine tended to be higher in the Test rats than in the Contrast rats (FIG. 4). More specifically, as compared with the OVX-treated Control rats, the OVX-treated Test rats shown in FIG. 4 had a critical rate of 0.1 or less, which indicates a tendency (corresponding to (1) in FIG. 4).

This suggests that the absorbability of isoflavone is improved in the composition of the invention than in a composition containing a non-fermented soybean product.

Accordingly, it is also suggested that the bone-metabolism-improving effects suggested by the composition of the invention result from the improvement in absorbability of isoflavone, and the usefulness of the composition of the invention is thus confirmed with high confidence.

### INDUSTRIAL APPLICABILITY

According to the method of the invention, the koji-making step can be shortened, and the production efficiency can be increased. Further, the novel miso-like food has strong umami, rich flavor, and strong taste. The novel miso-like food obtained by the invention has excellent taste advantages, and thus can be used for a seasoning or as a food. Specifically, extensive applications are expected such as to sources and like various seasonings, and soups, confectioneries, and like processed foods. Accordingly, the invention will be industrially extremely useful especially in the field of foods. Further, a composition containing the miso-like food of the invention having an ratio of isoflavone aglycone of 80 to 100% is effective in improving bone metabolism when taken as it is or as an eatable or a drinkable, and accordingly, such a composition provides a high health value and is expected to be useful in prevention or treatment of osteoporosis.

### FIG. 1

### Deoxypyridinoline in Urine

pmol/μmol creatinine

### Sham-Control

### OVX-Control

### OVX-Test

### FIG. 2

### Tibial Cancellous Bone Density

mg/cm³

### Sham-Control

### OVX-Control

### OVX-Test

### FIG. 3

### Ca Content of Tibia

mg/100 g BW

### Sham-Control

### OVX-Control

### OVX-Test

### FIG. 4

### Total Isoflavone in Urine

pmol/μmol creatinine

### OVX-Control

OVX-Test

1. A liquid koji, obtainable by culturing an aspergillus in 5 to 500 times its amount by weight of a lactic acid bacterium culture solution or a supernatant thereof.

2. A liquid koji according to claim 1, wherein said aspergillus is cultured at a temperature of 20 to 45° C. and for a time of 6 to 30 hours.

3. A liquid koji according to claim 1, wherein said lactic acid bacterium is a bacteriocin-producing bacterium.

4. A liquid koji according to claim 3, wherein said bacteriocin-producing lactic acid bacterium is a nisin-producing lactic acid bacterium.

5. A method of making a quick-brewed miso-like food, said method comprising:
(1) adding a liquid koji according to claim 1 to a food material;
(2) making koji in a hermetically sealed koji-making machine at 20 to 45° C. for 16 to 40 hours while successively or intermittently supplying disinfected air thereto, to obtain a koji;
(3) mixing a lactic acid bacterium culture solution or a supernatant thereof into said koji, to obtain a mixture;
(4) providing said mixture as it is or in paste form to obtain a moromi; and
(5) hydrolyzing said moromi substantially in the absence of salt.

6. The method of claim 5, wherein said (3) mixing a lactic acid bacterium culture solution or a supernatant thereof into said koji, to obtain a mixture, further comprises mixing therewith at least one kind of food material in an amount 0.01 to 50 times the weight of the koji.

7. The method of claim 5, wherein said lactic acid bacterium is a bacteriocin-producing bacterium.

8. The method of claim 7, wherein said bacteriocin-producing lactic acid bacterium is a nisin-producing lactic acid bacterium.

9. The method of claim 5, wherein said food material is at least one kind selected from the group consisting of rice, barley, soybean, soy germ, and mixtures thereof.

10. The method of claim 6, wherein said food material, which is added in said (3) mixing a lactic acid bacterium culture solution or a supernatant thereof into said koji, to obtain a mixture, is at least one kind selected from the group consisting of rice, barley, soybean, soy germ, and mixtures thereof.

11. The method of claim 5, wherein said food material is soybean.

12. The method of claim 6, wherein said food material, which is added in said (3) mixing a lactic acid bacterium culture solution or a supernatant thereof into said koji, to obtain a mixture, is rice.

13. A quick-brewed miso-like food, which is obtainable by a process according to claim 5.

14. A quick-brewed miso-like food according to claim 13, wherein the quick-brewed miso-like food has soy isoflavone aglycone form of 80 to 100%.

15. A bone-metabolism-improving composition, comprising a quick-brewed miso-like food having a soy isoflavone aglycone form of 80 to 100%.

16. A bone-metabolism-improving composition, comprising a quick-brewed miso-like food having isoflavone aglycone form of 80 to 100%, obtained by a process comprising:
(1) adding a liquid koji to soybean;
(2) making koji in a hermetically sealed koji-making machine at 20 to 45° C. for 16 to 40 hours while successively or intermittently supplying disinfected air thereto, to obtain a koji;
(3) mixing a lactic acid bacterium culture solution or a supernatant thereof into said koji, to obtain a mixture;
(4) providing said mixture as it is or in paste form to give a moromi; and
(5) hydrolyzing said moromi substantially in the absence of salt.

17. A bone-metabolism-improving composition according to claim 16, wherein said (3) mixing a lactic acid bacterium culture solution or a supernatant thereof into said koji, to obtain a mixture, further comprises mixing therewith at least one kind of food material in an amount 0.01 to 50 times the weight of the koji.

18. A bone-metabolism-improving composition according to claim 16, wherein said liquid koji is obtained by culturing an aspergillus in 5 to 500 times its amount by weight of a lactic acid bacterium culture solution or a supernatant thereof.

19. A bone-metabolism-improving composition according to claim 18, wherein said aspergillus is cultured at a temperature of 20 to 45° C. and for a time of 6 to 30 hours.

20. A bone-metabolism-improving composition according to claim 18, wherein said lactic acid bacterium is a bacteriocin-producing bacterium.

21. A bone-metabolism-improving composition according to claim 20, wherein said bacteriocin-producing lactic acid bacterium is a nisin-producing lactic acid bacterium.

22. A bone-metabolism-improving composition according to claim 16, wherein said food material is at least one kind selected from the group consisting of rice, barley, soybean, soy germ, and mixtures thereof.

23. A bone-metabolism-improving composition according to claim 17, wherein said food material, which is added in said (3) mixing a lactic acid bacterium culture solution or a supernatant thereof into said koji, to obtain a mixture, is at least one kind selected from the group consisting of rice, barley, soybean, soy germ, and mixtures thereof.

24. A bone-metabolism-improving composition according to claim 16, wherein said food material is soybean.

25. A bone-metabolism-improving composition according to claim 17, wherein said food material, which is added in said (3) mixing a lactic acid bacterium culture solution or a supernatant thereof into said koji, to obtain a mixture, is rice.

26. A bone-metabolism-improving composition according to claim 15, wherein said bone-metabolism-improving composition is eatable or drinkable.

27. A method of improving bone metabolism, comprising administering an effective amount of a bone-metabolism-improving composition according to claim 15 to a subject in need thereof.

28. A method of improving bone metabolism, comprising administering an effective amount of a bone-metabolism-improving composition according to claim 16 to a subject in need thereof.
